(19) United States
(12) Patent Application Publication
(10) Pub. No.: US 2005/0055097 A1
(43) Pub. Date: Mar. 10, 2005

(54) Minimally invasive modular support implant device and method

(75) Inventors: IIan Grunberg, Haifa (IL); Nissim Ohana, Raanana (IL); Asaf Ben-Arye, Zichron Yaakov (IL); Yuval Shezifi, Haifa (IL)

Correspondence Address: SALTAMAR INNOVATIONS, 30 FERN LANE, SOUTH PORTLAND, ME, 04106, US

(73) Assignee: EXPANDIS LTD. An Israeli corporation, Haifa (IL)

(21) Appl. No.: 10/879,845

(22) Filed: Jun. 29, 2004

Related U.S. Application Data
(63) Continuation-in-part of application No. 10/036,564, filed on Dec. 31, 2001, now abandoned.

(51) Int. Cl. A61F 2/44

(52) U.S. Cl. 623/17.11; 623/17.16

(57) ABSTRACT

Device and method are disclosed of a plate for use in conjunction with at least another one of a plurality of other plates in a modular reconstructing and supporting assembly for reconstructing and supporting a diseased or fractured bone or within a space previously occupied by a diseased intervertebral disc of a patient. The plate is sized small enough to be suitable for separate insertion into the bone or the space, preferably through a canule, and arrangement with the other plates adjacently one on top of the other to construct scaffolding, so as to provide a supporting prosthesis. In another preferred embodiment the plate has at least two substantially opposite aspects with interlocking features designed to facilitate interlocking of adjacent plates so as to prevent or restrain their sliding off each other.

### FIELD OF THE INVENTION

The present invention relates to orthopedic implants. More particularly, it relates to a device and method for modular implant, which provides support, and is introduced by minimal invasive procedure.

### BACKGROUND OF THE INVENTION

The spinal column serves as the support structure of the body, rendering the body its posture. Yet age, diseases and traumas hamper its completeness, and health, causing structural failures such as vertebral fractures, disc hernias, degenerative disk diseases, etc., resulting in pain and spinal instability, and even paralysis.

The adult vertebral column includes 26 vertebras (7 cervical, 12 thoracic, 5 lumbar, 1 sacrum and 1 coccyx) separated by intervertebral fibrocartilage discs.

A typical vertebra 10 (see FIG. 1), consists of two essential parts—an anterior segment, comprising the body 12, and a posterior part, comprising the vertebral or neural arch The vertebral arch consists of a pair of pedicles 14 and a pair of laminae 18, and supports seven processes—four articular, two transverse 16, and one spinous 20. The body and the vertebral arch define a foramen, known as the vertebral foramen 22. It should be noted that the vertebras' structure differs slightly according to the position on the spinal column (i.e. cervical, thoracic, and lumbar).

Among various vertebral column disorders, the typical ones include traumatic damages such as compression fractures, degenerative disc disease, disc hernias (ruptured or protruded disc), scoliosis (lateral bending of the vertebral column), kyphosis (exaggerated thoracic curvature), lordosis (exaggerated lumbar curvature), and spina bifidia (congenital incompletion of the closure of the vertebral column).

Various fixation, replacement and reconstructive solutions—both intravertebral and intervertebral were introduced in the past, some of which are mentioned hereinafter.

For example, U.S. Pat. No. 6,019,793 (Perren et al.), titled SURGICAL PROSTHETIC DEVICE, disclosed a surgical prosthetic device that is adapted for placement between two adjoining vertebrae for total or partial replacement of the disk from therebetween. The device has two plates with interior surfaces facing each other and being held at a distance by connecting means and exterior surfaces for contacting the end plates of the two adjoining vertebrae. The connecting means is made of a shape-memory alloy so that it is delivered to its destination cramped within a delivering tool and deploys once freed in position.

U.S. Pat. No. 5,423,816 (Lin) titled INTERVERTEBRAL LOCKING DEVICE disclosed an intervertebral locking device comprising one spiral elastic body, two bracing mounts and two sets of locking members. The two bracing mounts are fastened respectively to both ends of the spiral elastic body. The two sets of locking members are fastened respectively with the two bracing mounts such that each set of the locking members is anchored in one of the two vertebrae adjacent to a vertebra under treatment. The spiral elastic body and the vertebra under treatment evince similar elastic qualities, i.e. similar deflection characteristics. A plurality of bone grafts affinitive to the vertebra under treatment is deposited in the chambers of the spiral elastic body and in the spaces surrounding the spiral elastic body.

U.S. Pat. No. 5,423,817 (Lin) titled INTERVERTEBRAL FUSING DEVICE, teaches an intervertebral fusing device having a spring body portion interconnecting a first spiral ring mount and a second spiral ring mount. Each spiral ring mount has a spiralling projection on the outer surface. The spring body portion is defined by a plurality of spiral loops. The plurality of spiral loops and spiralling projection of the spiral ring mounts have a constant pitch. A mount cover and a head member are threaded into an internally threaded portion of a respective spiral ring mount thereby forming a chamber in which bone grafts affinitive to the cells and tissues of a vertebra may be housed. The spring body portion is similar in elasticity to the vertebra.

U.S. Pat. No. 5,306,310 (Siebels), titled VERTEBRAL PROSTHESIS, disclosed a prosthesis as a vertebral replacement element consisting of two helical strands, which may be screwed together to form a tubular structure. The implant is inserted between vertebrae and then slightly unscrewed until the desired height is reached. The helical strands consist of carbon fiber reinforced composite material.

U.S. Pat. No. 6,033,406 (Mathews) titled METHOD FOR SUBCUTANEOUS SUPRAFASCIAL PEDICULAR INTERNAL FIXATION disclosed a method for internal fixation of vertebra of the spine to facilitate graft fusion includes steps for excising the nucleus of an affected disc, preparing a bone graft, instrumenting the vertebrae for fixation, and introducing the bone graft into the resected nuclear space. Disc resection is conducted through two portals through the annulus, with one portal supporting resection instruments and the other supporting a viewing device. The fixation hardware is inserted through small incisions aligned with each pedicle to be instrumented. The hardware includes bone screws, fixation plates, engagement nuts, and linking members. In an important aspect of the method, the fixation plates, engagement nuts and linking members are supported suprafascially but subcutaneously so that the fascia and muscle tissue are not damaged. The bone screw is configured to support the fixation hardware above the fascia. In a further aspect of the invention, a three-component dilator system is provided for use during the bone screw implantation steps of the method.

Generally, these described methods and devices are very invasive and involve massive surgical involvement.

Minimally invasive system is described in U.S. Pat. No. 6,248,110 (Reiley et al.) titled SYSTEMS AND METHODS FOR TREATING FRACTURED OR DISEASED BONE USING EXPANDABLE BODIES. Systems and methods are disclosed for treating fractured or diseased bone by deploying more than a single therapeutic tool into the bone. In one arrangement, the systems and methods deploy an expandable body in association with a bone cement nozzle into the bone, such that both occupy the bone interior at the same time. In another arrangement, the systems and methods deploy multiple expandable bodies, which occupy the bone interior volume simultaneously. Expansion of the bodies form cavity or cavities in cancellous bone in the interior bone volume. Use of expandable balloon is taught, which serves for reconstruction of collapsed bone. In order to fill the space created and provide stabilization to the bone, insertion of polymethylmethacrylate cement that hardens and stiffens is required.

The above-mentioned fixation and support solutions (and others) all introduce mechanical structures to gain support and/or fixation. All these devices are surgically placed in the desired position. Some of them require a major surgical operation involving major invasive actions. Polymethylmethacrylate (PMMA) cement is not suitable for insertion in young people, since it tends to loosen, hence the fixation is jeopardized. In addition, it may involve side effects such as spinal cord injuries, radiculopathies, and cement leakage. Furthermore, the cement is hard to control and maintain during insertion because of its fluidic nature, hardening process, and consistency.

### BRIEF DESCRIPTION OF THE INVENTION

It is the purpose of the present invention to provide a minimally invasive method and device for reconstructing and supporting a fractured or diseased bone, preferably a fractured or diseased vertebra. In an alternative embodiment of the present invention the method and device disclosed herein are aimed at providing support within a space previously occupied by diseased bone or intervertebral disc that has been completely or partially removed.

It is therefore provided, in accordance with a preferred embodiment of the present invention, a modular reconstructing and supporting assembly for reconstructing and supporting a diseased or fractured bone or within a space previously occupied by a diseased intervertebral disc, the assembly comprising: a plurality of plates adapted to be cooperatingly inserted into the bone, at least one of said plates arranged adjacently to another plate within said bone or space, to construct scaffolding for forming a supporting prosthesis.

Furthermore, in accordance with a preferred embodiment of the present invention, at least one of said plates having at least two substantially opposite aspects with interlocking features designed to facilitate interlocking of adjacent plates so as to prevent or restrain relative movement therebetween.

Furthermore, in accordance with a preferred embodiment of the present invention, the opposite aspects of the plate are inclined with respect to each other.

Furthermore, in accordance with a preferred embodiment of the present invention, one of said aspects is provided with at least one longitudinal protrusion and the opposite aspect is provided with at least one corresponding longitudinal recess designed to receive a longitudinal protrusion of an adjacent plate.

Furthermore, in accordance with a preferred embodiment of the present invention, one aspect is provided with at least one lateral protrusion and the opposite aspect is provided with at least one corresponding lateral recess designed to accommodate a lateral protrusion of an adjacent plate.

Furthermore, in accordance with a preferred embodiment of the present invention, one aspect is provided with at least one longitudinal protrusion and at least one lateral protrusion and the opposite aspect is provided with at least one corresponding longitudinal recess designed to accommodate a longitudinal protrusion of an adjacent plate, and with at least one corresponding lateral recess designed to accommodate a lateral protrusion of an adjacent plate.

Furthermore, in accordance with a preferred embodiment of the present invention, the interlocking features include at least one recess on one aspect and at least one corresponding projection on the other aspect, so that the projection of one plate is accommodatable in the recess of an adjacent plate.

Furthermore, in accordance with a preferred embodiment of the present invention, the recess further comprises a rim adapted for retaining the projection of an adjacent plate, for preventing or restraining relative displacement therebetween.

Furthermore, in accordance with a preferred embodiment of the present invention, the rim extends along a portion of the circumference of the recess, allowing leveled sliding in of the projection of the adjacent plate.

Furthermore, in accordance with a preferred embodiment of the present invention, at least one of said plurality of plates is curved.

Furthermore, in accordance with a preferred embodiment of the present invention, the plate is provided with at least one tapered end, for facilitating plate guidance and positioning between two adjacent plates.

Furthermore, in accordance with a preferred embodiment of the present invention, the tapered end is in the form of a wedge.

Furthermore, in accordance with a preferred embodiment of the present invention, the plate is made from or coated with biocompatible material.

Furthermore, in accordance with a preferred embodiment of the present invention, the plate is made from material selected from a group consisting of metal, titanium, titanium alloy, stainless steel alloys, steel 316, processed foil, hydroxyapatite, material coated with hydroxyapetite, plastics, silicon, composite materials, carbon-fiber, hardened polymeric materials, polymethylmetacrylate (PMMA), ceramic materials, coral material or a combination thereof.

Furthermore, in accordance with a preferred embodiment of the present invention, at least one of said plates is coated with hydroxyapetite

Furthermore, in accordance with a preferred embodiment of the present invention, the plate is covered with a bone growth encouraging substance.

Furthermore, in accordance with a preferred embodiment of the present invention, said plate being is coated with bone morphogenic protein.

Furthermore, in accordance with a preferred embodiment of the present invention, wherein the plate is coated with medication.

Furthermore, in accordance with a preferred embodiment of the present invention, said plate is coated with a substance selected from the group consisting of antibiotics, slow releasing medication, chemotherapic substances, or a combination thereof.

Furthermore, in accordance with a preferred embodiment of the present invention, the plate comprises non-ferrous material.

Furthermore, in accordance with a preferred embodiment of the present invention, the plate is coated with lubricating material to facilitate sliding the plates into a desired position.

Furthermore, in accordance with a preferred embodiment of the present invention, the plate is coated with coating materials that sublime or react to form a solid conglomerate.

Furthermore, in accordance with a preferred embodiment of the present invention, the plate is substantially disc-shaped.

Furthermore, in accordance with a preferred embodiment of the present invention, the plate is provided with a groove adapted to be engaged by a holding tool.

Furthermore, in accordance with a preferred embodiment of the present invention, the assembly further comprises a pin protruding from at least one of said plates, to facilitate placement of said plate.

Furthermore, in accordance with a preferred embodiment of the present invention, at least one of said plates having a rough external surface.

Furthermore, in accordance with a preferred embodiment of the present invention, the plate is provided with a plurality of substantially parallel grooves, so as to facilitate sliding of one plate adjacent another such plate.

Furthermore, in accordance with a preferred embodiment of the present invention, a bore is provided on the plate to facilitate hooking of the plate onto an introducing tool and releasing it when it is positioned at a desired location.

Furthermore, in accordance with a preferred embodiment of the present invention, the plate is provided with a bore with open rim.

Furthermore, in accordance with a preferred embodiment of the present invention, the assembly further comprises a lead in the form of a conduit with a proximal end and a distal end, the conduit having an inlet at the proximal end and two substantially opposite slits about the distal end, so that when plates are inserted through the inlet and advanced towards the distal end, some plates protrude out of the slits to form the plate assembly.

Furthermore, in accordance with a preferred embodiment of the present invention, the lead is provided with thread at its proximal end.

Furthermore, in accordance with a preferred embodiment of the present invention, the thread is internal.

Furthermore, in accordance with a preferred embodiment of the present invention, the thread is external.

Furthermore, in accordance with a preferred embodiment of the present invention, a packing strip is provided in the lead to hold the plate assembly together.

Furthermore, in accordance with a preferred embodiment of the present invention, the assembly is further provided with a stopper in the form of a plug that plugs into the lead holding sides of the packing strap against the lead so as to lock the strap in position.

Furthermore, in accordance with a preferred embodiment of the present invention, the lead is provided with spaces designed to encourage bone growth into it.

Furthermore, in accordance with a preferred embodiment of the present invention, the slits are carved into the lead in an entwining form so as to produce portions that may bulge outwardly, for holding the plate assembly when erected.

Furthermore, in accordance with a preferred embodiment of the present invention, the entwined form consists of a curved strip.

Furthermore, in accordance with a preferred embodiment of the present invention, two straps are further provided within the lead, long enough so that when the plate assembly is erected, one strap covers the plate assembly from one side whereas the other strap closes on the plate assembly from another opposite side, portions of the straps overlapping at the distal end.

Furthermore, in accordance with a preferred embodiment of the present invention, the assembly is further provided with a crampable deployable cage for hosting the plate assembly when erected.

Furthermore, in accordance with a preferred embodiment of the present invention, the cage is a stent.

Furthermore, in accordance with a preferred embodiment of the present invention, the assembly is provided in a cartridge.

Furthermore, in accordance with a preferred embodiment of the present invention, the cartridge comprises a housing for hosting a plurality of plates stacked one on top of each other, with an inlet and outlet, the inlet and outlet substantially opposing each other, and a resilient member for pressing plates against the outlet so as to allow convenient drawing of a plate from the cartridge.

Furthermore, in accordance with a preferred embodiment of the present invention, the cartridge comprises an elongated housing for hosting a plurality of plates arranged in a line, with an adjacent introducing duct, the cartridge provided with an opening into the introducing duct so that one plate at a time may be inserted into the introducing duct and advanced through the duct to a target location using an introducing tool.

Furthermore, in accordance with a preferred embodiment of the present invention, there is provided a lead device for introducing and supporting a plate assembly made of stacked plates, the lead comprising a conduit with a proximal end and a distal end, the conduit having an inlet at the proximal end and two substantially opposite slits about the distal end, so that when plates are inserted through the inlet and advanced towards the distal end, some plates protrude out of the slits to form the plate assembly.

Furthermore, in accordance with a preferred embodiment of the present invention, the lead is further provided with a tiltable plate anchorage for anchoring plates to it for improved stability of the plate assembly.

Furthermore, in accordance with a preferred embodiment of the present invention, the tiltable plate anchorage is in the form of a blade having an elongated end presenting a T-shaped cross-section, with a narrow portion and a wider portion, the blade capable of being initially advanced through the lead in a horizontal position, and as it reaches the distal portion it is capable of flipping to an upright vertical position.

Furthermore, in accordance with a preferred embodiment of the present invention, a central portion of the elongated end presenting a T-shaped cross-section is tapered so as to allow plates having an open bore at their end to be hooked onto the end, and when the plates shift upwards or downwards along the anchorage blade, the wider portion substantially occupies the bore, so that the plate cannot be released from the anchorage blade, thus providing additional stability to the plate assembly.

Furthermore, in accordance with a preferred embodiment of the present invention, there is provided a delivery tool for delivering a device as claimed in Claim 45 into a diseased or fractured bone or within a space previously occupied by a diseased intervertebral disc, the delivery tool comprising two coaxial pipes, one internal pipe and one external pipe, the external pipe adapted to be shifted over the internal pipe so as to cover the latter or expose it, so that an engagement means located at a distal tip of the internal pipe is engaged when the external pipe covers the distal end of the internal pipe and disengaged when the distal end of the internal pipe is exposed.

Furthermore, in accordance with a preferred embodiment of the present invention, the internal pipe is provided at the distal end with a recess of a predetermined shape so as to accommodate a matching protrusion of the device thus coupling the device to the delivery tool.

Furthermore, in accordance with a preferred embodiment of the present invention, there is provided a spacing tool for spacing and evaluating the spacing between adjacent plates of the assembly claimed in Claim 1, the spacing tool comprising a rod with a tapered end.

Furthermore, in accordance with a preferred embodiment of the present invention, the tapered end is provided with a wedge.

Furthermore, in accordance with a preferred embodiment of the present invention, a packing strap is provided to hold the plate assembly together when erected.

Furthermore, in accordance with a preferred embodiment of the present invention, there is provided a plate for use in conjunction with at least another one of a plurality of other plates in a modular reconstructing and supporting assembly for reconstructing and supporting a diseased or fractured bone or within a space previously occupied by a diseased intervertebral disc of a patient, the plate sized small enough to be suitable for separate insertion into the bone or the space and arrangement with the other plates adjacently to construct scaffolding, so as to provide a supporting prosthesis.

Furthermore, in accordance with a preferred embodiment of the present invention, there is provided a method for reconstructing and supporting within a diseased or fractured bone or within a space previously occupied by a diseased intervertebral disc the method comprising:
inserting a plurality of plates into the bone
arranging said plates adjacent one another, within the bone or space, to construct a support scaffolding.

Furthermore, in accordance with a preferred embodiment of the present invention, the method further comprises the steps of delivering each plate separately into the bone using low profile delivery means, through a small incision in the skin of the patient, and arranging adjacent plates on top of each other.

Furthermore, in accordance with a preferred embodiment of the present invention, the delivery means comprises a canula and a rod with which the plates are each advanced through the canula.

Furthermore, in accordance with a preferred embodiment of the present invention, the rod is provided with holding means to hold the plates.

Furthermore, in accordance with a preferred embodiment of the present invention, the bone is a vertebra and the plates are inserted through a bore drilled into the body of the vertebra through a pedicle of the vertebra.

Furthermore, in accordance with a preferred embodiment of the present invention, the diameter of the bore is in a range between 4 to 8 mm.

Furthermore, in accordance with a preferred embodiment of the present invention, at least one of said plates has at least two substantially opposite aspects with interlocking features designed to facilitate interlocking of adjacent plates, for preventing or restraining relative displacement therebetween.

Furthermore, in accordance with a preferred embodiment of the present invention, one aspect is provided with at least one longitudinal protrusion and the opposite aspect is provided with at least one corresponding longitudinal recess designed to accommodate the longitudinal protrusion of an adjacent plate.

Furthermore, in accordance with a preferred embodiment of the present invention, one aspect is provided with at least one lateral protrusion and the opposite aspect is provided with at least one corresponding lateral recess designed to accommodate the lateral protrusion of an adjacent plate.

Furthermore, in accordance with a preferred embodiment of the present invention, one aspect is provided with at least one longitudinal protrusion and at least one lateral protrusion and the opposite aspect is provided with at least one corresponding longitudinal recess designed to accommodate the longitudinal protrusion of an adjacent plate, and with at least one corresponding lateral recess designed to accommodate the lateral protrusion of an adjacent plate.

Furthermore, in accordance with a preferred embodiment of the present invention, the interlocking features include at least one recess on one aspect and at least one corresponding projection on the other aspect, so that the projection of one plate is accommodated in the recess of an adjacent plate.

Furthermore, in accordance with a preferred embodiment of the present invention, at least one of said plurality of plates is provided with at least one tapered end, to facilitate positioning the plate between two adjacent plates.

Furthermore, in accordance with a preferred embodiment of the present invention, at least one of said plurality of plates is substantially disc-shaped.

Furthermore, in accordance with a preferred embodiment of the present invention, at least one of said plurality of plates is further provided with a protruding pin, adapted to facilitate holding the plate by a delivering tool.

Furthermore, in accordance with a preferred embodiment of the present invention, the plates are inserted bilaterally constructing at least two scaffolding structures within a vertebral body.

Furthermore, in accordance with a preferred embodiment of the present invention, the plates are positioned one on top of the other.

Furthermore, in accordance with a preferred embodiment of the present invention, there is provided a method for reconstructing and supporting within a diseased or fractured bone or within a space previously occupied by a diseased intervertebral disc the method comprising:
providing a plurality of plates adapted to be separately inserted into the bone and arranged adjacently within the bone or space to construct scaffolding for providing support;
providing delivery means having low profile for delivering each plate through a small incision in the skin of the patient and into the bone or disc;
delivering each plate separately into the bone;
arranging the plates one adjacent the other.

Other aspects and features of the present invention are described in detail hereinafter.

### BRIEF DESCRIPTION OF THE FIGURES

In order to better understand the present invention, and appreciate its practical applications, the following Figures are provided and referenced hereinafter. It should be noted that the Figures are given as examples only and in no way limit the scope of the invention.

FIG. 1 illustrates an elevated view of two alternative preferred embodiments of a vertebral modular support implant device in accordance with preferred embodiments of the present invention, implanted in the body of a vertebra.

FIGS. 2 to 5 illustrate various stages of intra-vertebral implant surgical implantation.

FIG. 2 illustrates the pedicular access into the body of the vertebra using a guide and a drill.

FIG. 3 illustrates the insertion of the first of a series of plates making up the modular support structure of the present invention through a deployed canula, using a delivery tool.

FIG. 4 illustrates the insertion of yet another plate between previously deployed plates.

FIG. 5 illustrates the final position of the vertebral modular support implant device within the body of the vertebra.

FIGS. 6a-6d illustrate several optional configurations for a single plate.

FIG. 7 illustrates another alternative configuration for a single plate—in the form of a disc.

FIG. 8 illustrates yet another alternative configuration for a single plate—in the form of a disc provided with a protruding pin.

FIG. 9a illustrates another alternative configuration for a single plate with grooves and a closed bore at one end.

FIG. 9b illustrates another alternative configuration for a single plate, with grooves and an open bore at one end.

FIG. 10 illustrates a lead for deploying a plate assembly in accordance with the present invention, with a back flange.

FIG. 11 illustrates a shortened lead for deploying a plate assembly in accordance with the present invention.

FIG. 12 illustrates a lead for deploying a plate assembly in accordance with the present invention, with internal thread.

FIG. 13 illustrates a lead for deploying a plate assembly in accordance with the present invention, with external thread.

FIG. 14 illustrates a plate assembly with a packing strip for packing the plate assembly in accordance with the present invention.

FIG. 15 illustrates a lead for deploying a plate assembly in accordance with the present invention, with a packing strip.

FIG. 16 illustrates a lead for deploying a plate assembly in accordance with the present invention, with spaces provided on the body of the lead for enhanced bone growth around the lead.

FIG. 17a illustrates a lead for deploying a plate assembly in accordance with the present invention, with integral deployable packing strip.

FIG. 17b illustrates another lead for deploying a plate assembly in accordance with the present invention, with integral deployable packing strip.

FIG. 18 illustrates a sectioned view of a lead for deploying a plate assembly in accordance with the present invention, with deployable and partially overlapping packing strips.

FIG. 19 illustrates a sectioned view of a lead for deploying a plate assembly in accordance with the present invention, with a packaging strip and a stopper. FIG. 20 illustrates a lead for deploying a plate assembly in accordance with another preferred embodiment of the present invention, with a deployable cage.

FIG. 21 illustrates a plate assembly with a deployable cage in the deployed state.

FIG. 22a illustrates a lead for deploying a plate assembly in accordance with another preferred embodiment of the present invention, with a tiltable plate anchorage.

FIG. 22b illustrates a side view of the lead of FIG. 22a with a plate anchored to the tiltable plate anchorage.

FIG. 22c illustrates a side view of a portion of the lead of FIG. 22a with the front side of the lead missing to allow understanding of how the plates anchor to the tiltable plate anchorage.

FIG. 23 illustrates a plate cartridge with vertically mounted plates in accordance with a preferred embodiment of the present invention.

FIG. 24 illustrates another plate cartridge with plates arranged in a column, and provided with an introducing duct.

FIG. 25 illustrates a delivery tool in accordance with a preferred embodiment of the present invention, with yet another preferred embodiment of a lead for deploying a plate assembly mounted on its tip.

FIG. 26 illustrates a spacing tool for providing room and controlling the alignment of the plates of a plate assembly in accordance with a preferred embodiment of the present invention.

### DETAILED DESCRIPTION OF THE INVENTION AND FIGURES

The present invention relates to repair of damaged bones, primarily to damaged or diseased vertebras, and in particular it appeals in relation to compressed fractures of the body of the vertebra, caused by trauma or related to osteoporosis. Similarly, although a slightly different approach is required, the present invention may relate to fixation of the spine, in cases of degenerative intervertebral disc disease, where the structure disclosed herein may serve as intervertebral fixation device similar to an intervertebral cage.

In accordance with a preferred embodiment, the vertebral reconstruction and support implant method is a minimally invasive surgical method, involving inserting plates, through a small incision in the skin and surrounding muscle tissue, using low profile (i.e. narrow) delivery tools, into the vertebral body or into the inter-vertebral disk area, in order to reconstruct the original anatomic structures. The method fits in particular the treatment of collapsed vertebral body or degenerative disk space. After using it for reconstruction of the anatomical structure of the vertebral body, this assembly further functions as a prosthesis, which supports the vertebra internally (within the cortex) or externally (intervertebrally), substantially maintaining the normal original shape of the vertebra and the spinal structure.

A typical vertebral modular support implant system comprises a plurality of plates, capable of being mounted one on top of the other or next to each other in a lateral adjacent configuration and staying secured in that position so as to present a modular scaffolding structure.

The shape of these plates is designed to allow precise sliding of every plate on top, bellow, or next to the other. In a preferred embodiment of the present invention, in order to accomplish that aim, a recess and corresponding protrusion design is used. It is very desirable that the plate design ensures the prevention or substantial restraining of the plates from sliding off each other.

In order to place each of the plates in the desired position and location a preferable delivery system is used. The characteristics of such system are explained hereinafter.

Insertion and placement of the plates one on top of the other or next to the other creates a wall or stent, that reconstructs and supports the anatomic structure of the organ treated.

The present invention, although not limited to this purpose only, presents a system and method that is particularly suited for treating fractured and compressed bones and more particularly compression fracture of vertebral bodies. In an alternative embodiment of the present invention it is suggested to implement the modular support implant device for treating a degenerative disc disease, by replacing the diseased disc or most of it and positioning the modular support implant device intervertebrally.

The implementation of the present invention requires minimally invasive surgery that significantly reduces damage to adjacent tissues existing around the treated organ, and is usually much faster to perform, reducing surgical procedure time, hospitalization and recovery time, and saving costs.

An important aspect of the present invention is using a method and device (modular plate construction in our case) to reconstruct an anatomic structure. Then, the same device, left as an implant on location, serves as a fixation and a prosthesis device.

The above-mentioned concept brings about several additional advantages and properties that can be characterized as follows:

The present invention introduces a minimally invasive method and approach for treating the affected bone, hence causing minimal damage to adjacent tissues and anatomic structures. It then, uses a prosthesis built from plates to reconstruct a compressed bone back to its normal structure, forming a scaffolding structure to support the vertebral body or other structure treated. This is done while saving essential surrounding ligaments, muscles, and other tissues responsible for providing the stabilization of the vertebral column.

Primarily the purpose of the present invention is to provide a solution for compressed or burst fractured vertebras. The present invention has a real appeal for osteoporosis and trauma related compression fractures. However, it is asserted that the present invention may be used to treat degenerative disc diseases by replacing an ill intervertebral disc and enhancing spine fixation.

In a preferred embodiment of the present invention reconstruction of the vertebral body is achieved by bilateral insertion of plates through both pedicles, in two sets, each set arranged one on top of the other, or both sets in an alternating order, to create a double wall-like prosthesis. In other words, jacking the collapsed end-plates of the vertebra is achieved by gradual expansion of the implant, constructed from the inserted plates. In a preferred embodiment of the invention, both sets are interconnected at one end to present a corner or a united bond. In another preferred embodiment (for example intervertebral implementation) it may be possible to build more than two scaffoldings (i.e. construct more than two such supporting structures).

Building an implant inside the treated area is a novel concept and treatment technique., Driven from the need to cause minimum damage to tissue while operating on a patient, the method employs minimally invasive technique. Other operation techniques of vertebral bones require open and prolonged surgery, hence creating damage to healthy tissue.

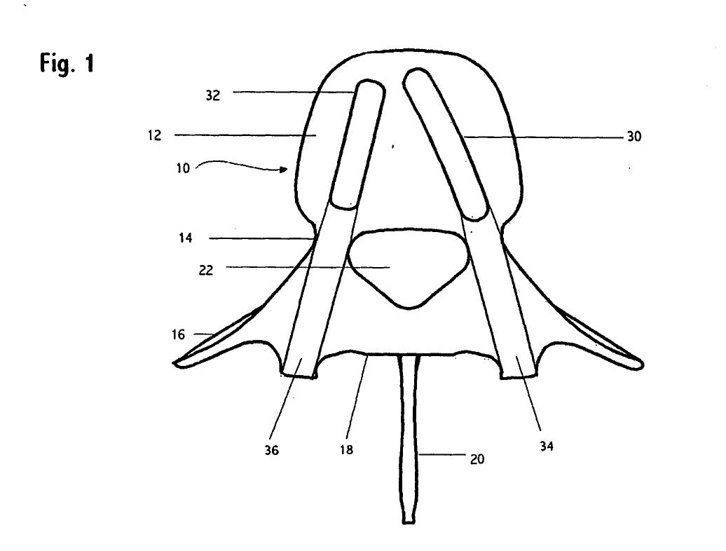

Reference is now made to FIG. 1 illustrating an elevated view of two embodiments of a vertebral modular support implant device in accordance with the present invention, implanted in the body of a vertebra.

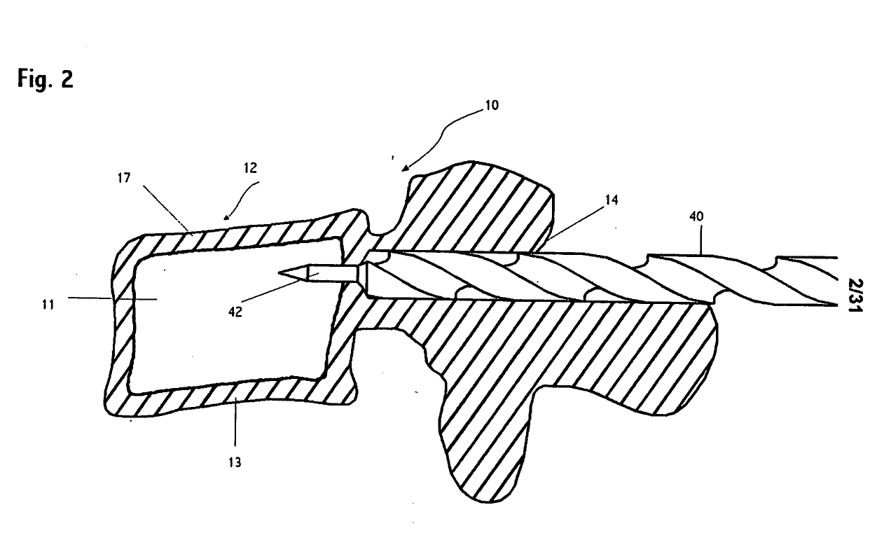

Into the damaged vertebral body 12 at least one vertebral modular implant support device is inserted and erected. In FIG. 1 two such structures are shown—a straight structure 32 and a curved structure 30. A curved structure provides better stabilization although a straight structure may also be considered (or even preferred for various reasons). The vertebral modular implant support structure is made of a plurality of plates, mounted one on top of the other until reaching a desired height, in order to provide support for the bone—the body cortical end-plate bones (13 and 17—see FIGS. 2-5) in the case of a vertebra—from within the body. The plates are inserted into the vertebral body via a drilled bore (34 for structure 30 or 36 for structure 32) through the pedicle's cortex 14. Typically, the diameter of the bore is anticipated to range between 4 to 8 mm according to the size of the vertebra and its pedicle (but the present invention is not limited to these measurements).

A preferred method of deployment of the vertebral modular implant support device is hereby explained with reference to FIGS. 2 to 5, illustrating various stages of intra-vertebral implant surgical implantation.

The vertebra is accessed in a minimally invasive manner. A guide 42 (see FIG. 2) is inserted through a small incision in the patient's skin and through the muscle tissue towards the vertebra, approaching it in the direction of one of the pedicles. The pedicle 14 is chosen to be the one nearest the desired target position of the vertebral modular implant support assembly. Note that it is recommended to employ the modular implant support device bilaterally, i.e. deploy two such modular constructions through both pedicles. However deployment of the implant support device through only one pedicle is also possible and is covered by the scope of the present invention. The guide is provided with a tapered distal end and is used to puncture and penetrate; pedically the vertebra into the vertebral body.

Once the guide is positioned, a drill 40 provided with a lumen extending through it, is advanced over the guide, which passes through the lumen. It is used to drill a bore through the pedicle into the vertebral body 12. The upper 17 and lower 13 vertebra end-plates are made from cortical bone, whereas the inside 11 of the body is of cancellous or spongeous bone. The bore is extended into the inside of the vertebral body.

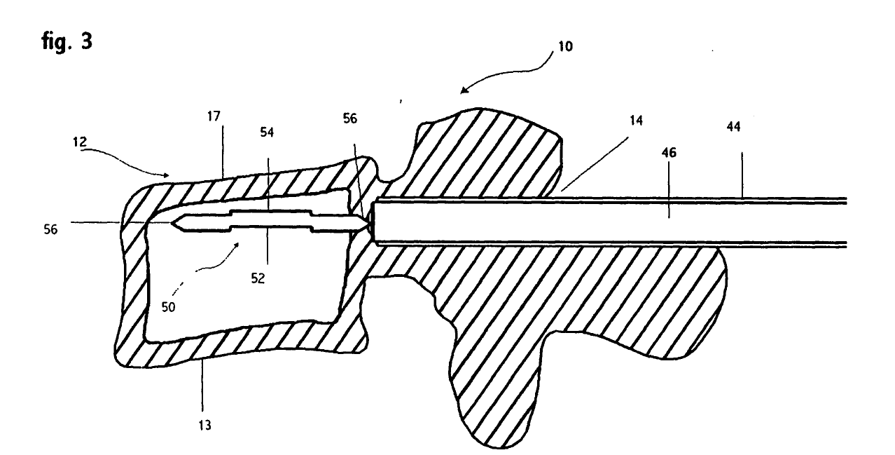

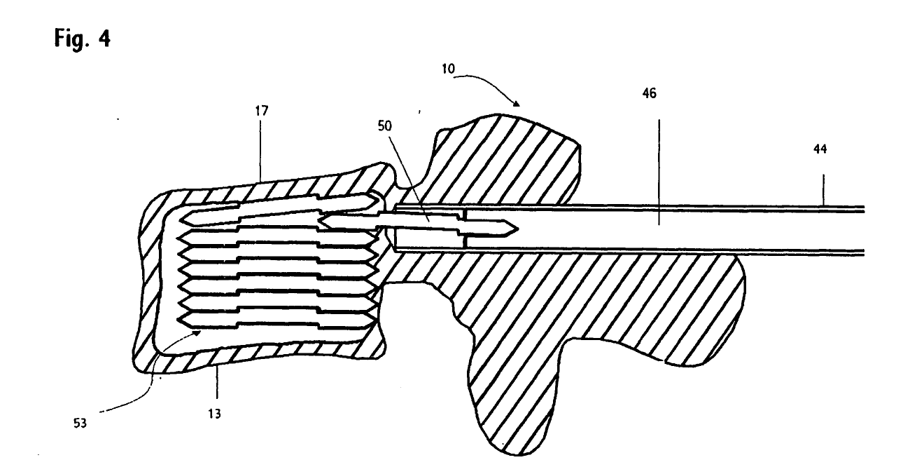

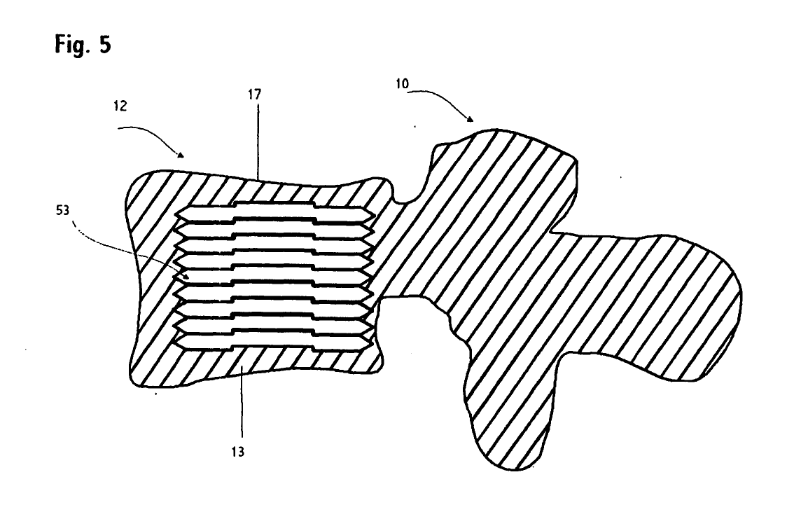

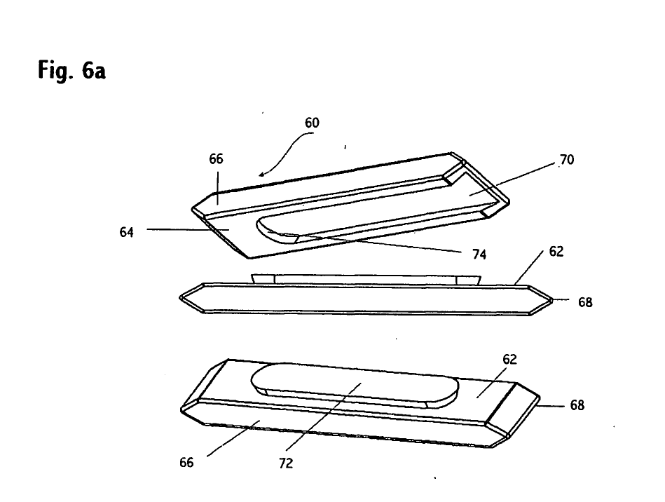

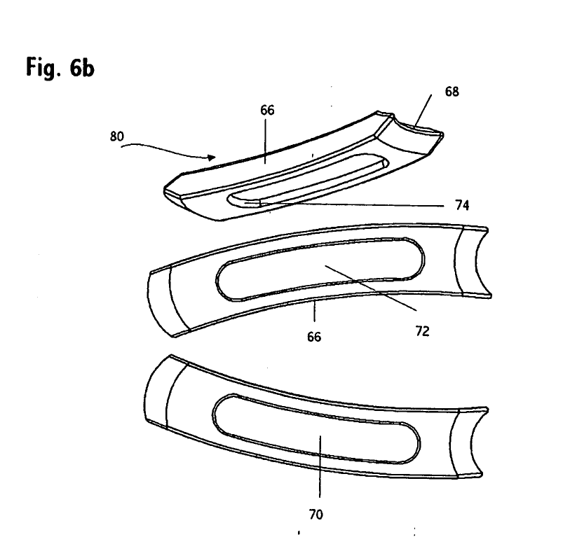

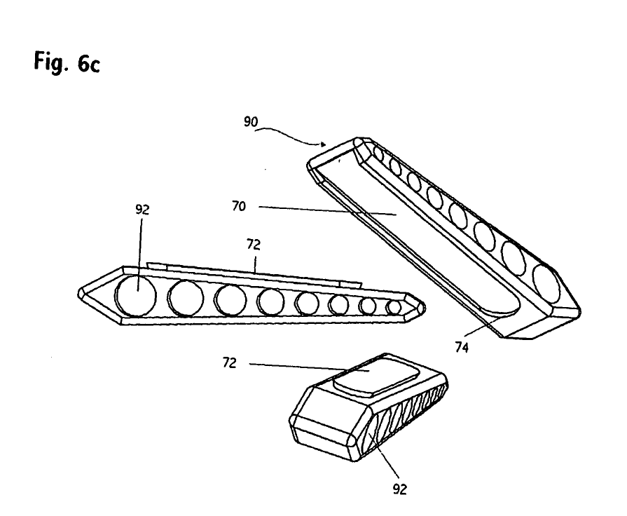

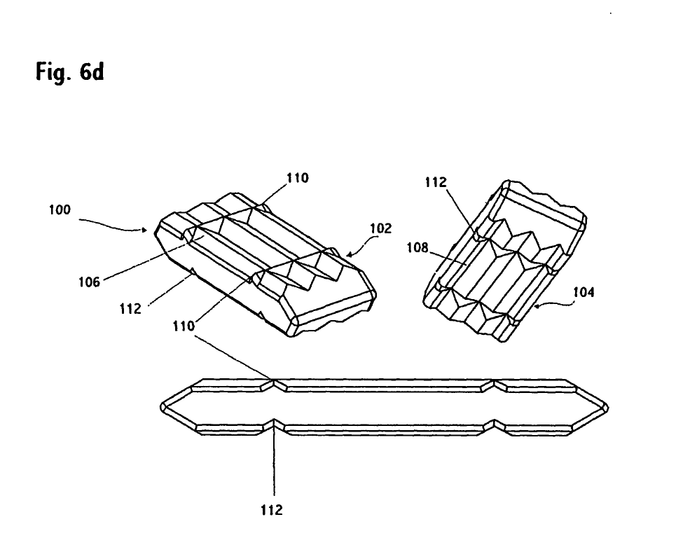

After the bore is drilled, the drill is removed and a canula 44 (see FIG. 3) is guided over the guide 42 through the bore (when in position the guide is removed). Optionally the canula may be provided with external thread for screwing it into the drilled bore and achieving enhanced stability. A first plate 50 is inserted through canula 44, advanced by a delivery tool 46, which may be a tube, a rod or similar elongated tool, until it is fully inside the body, and positioned in the target location. The delivery tool 46 may include a holding facility at its distal tip for holding the plate and release it on location, or simply push the plate to advance it. The plate 50 is designed to form a building block in a modular structure configuration that is to serve as a support structure within the vertebral body. In one preferred embodiment of the plate in accordance with the present invention, the plate is elongated, having at least one—in this case two—wedged ends 56, so as to allow inserting the plate between adjacent plates (see also FIGS. 4 and 5). The upper surface of the plate, is provided with projection 54 that fits into a corresponding recess 52 of an adjacent plate, so as to enhance the stability of the modular structure. Optional design examples are presented in FIG. 6. Preferably, imaging techniques such as fluoroscopy or navigation systems are used in order to facilitate correct positioning of the plates, however other visual or tactile means may be employed.

Similarly, more plates 50 (see FIG. 4) are inserted into the body. Note that subsequently inserted plates are guided into position on top (or bottom, or side by side) of the adjacent plate due to the nature of the topography of the adjacent plates, i.e. the indented surface on one plate and the corresponding protrusion of the adjacent plate.

More plates are inserted and guided into the vertebral modular implant support assembly 53 (see FIG. 5) that is formed within the vertebral body 12, until a desired height is reached, facilitating jacking of the vertebral end-plates (lower end-plate 13 and upper end-plate 17) further apart to the original (or new desired) position, preventing the collapse of these end-plate walls inwardly. At that stage, the delivery tool and the canula are removed. In the natural healing process of the bone, the bore is filled with new bone matter, and the vertebral modular implant support assembly becomes embedded within the bone, which secures its position and stability.

Note that the present invention may be implemented for providing support to enhance fixation in an intervertebral space previously occupied by a disc. The delivery method may be any minimally invasive approach. Currently there are some minimally invasive approaches for example endoscopic nucleotomy, etc. Such methods may be used, possibly with minor adjustments, in conjunction with the present invention.

FIGS. 6a-6d illustrate several optional configurations for a single plate. Each Figure illustrates three plates of the same sort, viewed from different angles. The plate of the present invention generally comprises a plate having at least two substantially opposite aspects designed to interlock. For the purpose of the present invention “interlocking” means any interlocking mechanism including various types of joining (such as binding, clasping, gripping, interlocking, uniting, hooking etc.), and also partial hooking that merely enhances the stability of the mounted plates.

Plate 60 in accordance with a preferred embodiment of the present invention, shown in FIG. 6a comprises an elongated flat plate having two generally opposite aspects—one aspect being the top surface 62 and the opposite aspect being the bottom surface 64 of the plate, and two narrower side aspects 66. The far ends 68 of the plate are wedged (or tapered) so as to allow guiding the plate through and positioning it between two adjacent plates, by separating them apart and sliding therebetween. On the bottom surface 64 a recess 70 is provided, corresponding to a projection 72 on the top surface 62, so as to allow sliding of two adjacent plates—one on top of the other, and preventing their sliding off each other. It is optional to provide a rim 74, either partially, allowing leveled sliding in of the projection of the adjacent plate, as shown in FIG. 6a, or about the entire recess, as shown in FIG. 6b, that serves to retain the projection of the adjacent bottom plate, preventing or at least limiting longitudinal relative displacement between adjacently mounted plates. In the plate shown in FIG. 6b the lateral aspects 66 are mutually curved in a configuration that is aimed at enhanced stability.

In accordance with another preferred embodiment of the present invention, the plate 90 shown in FIG. 6c is aimed at providing inclined support, its top and bottom surfaces inclined with respect to each other rendering one end higher than the other, so that by mounting several plates on top of each other, the total angle of inclination of the vertebral modular implant support assembly is the sum of inclination angles of each of the plates. The plate is provided with a plurality of bores 92, extended laterally across the plate, which may serve for enhancing bone ingrowth and thus enhance incorporation of the implant with the bone structure.

In accordance with another preferred embodiment of the present invention, the plate 100 shown in FIG. 6d the plate is grooved. The top surface 102 is provided with longitudinal protrusions 106 (at least one) and optionally two lateral protrusions 110 (at least one), whereas the bottom surface 104 is provided with corresponding longitudinal recesses 108 designed to accommodate the longitudinal protrusions of the adjacent plate, and two lateral recesses 112 designed to accommodate the lateral protrusions of the adjacent plate. This configuration has particular enhanced stability, both in lateral and longitudinal aspects.

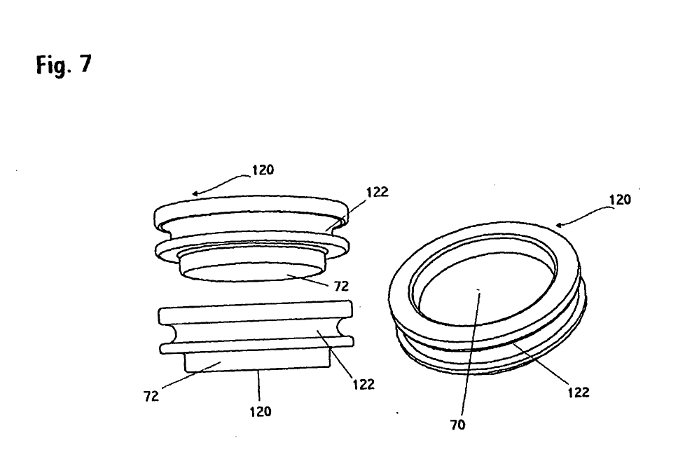

FIG. 7 illustrates yet another alternative embodiment of the plate (showing it in three views), in the form of a disc. The plate 120 is shaped like a disc, with a round protrusion 72 on one aspect (here on the bottom) and a corresponding recess 70 on the other opposite aspect (on top). An optional groove 122 is provided around the lateral aspect of the disk around its perimeter for holding the plate by means of a wire or string that may be removed or discarded once the plate is in position.

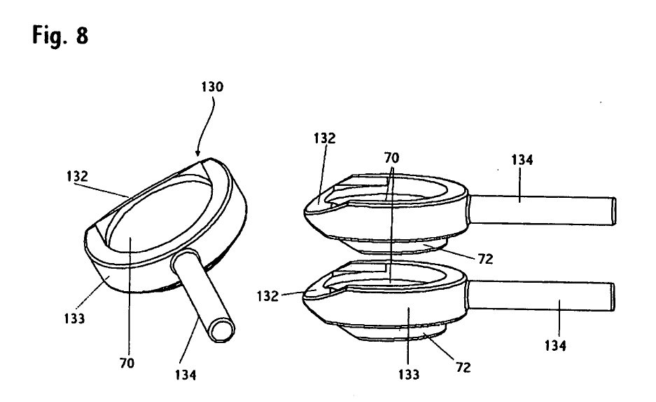

FIG. 8 illustrates yet another alternative embodiment of the plate (showing it in three views). The plate 130 consists of two general parts—a disc 133 and a pin 134, coupled to the disc protruding laterally. The pin 134 is provided as a handle (by a delivering tool) so as to ensure its safe guiding to its target position. The disc has a protrusion 72 and an opposite corresponding recess 70 and is tapered 132 on the side opposite to the pin. The protruding pin may protrude in various directions (i.e. not only laterally), provided it is possible to guide it through the guiding canula, or possible to achieve its final positioning by other delivery means.

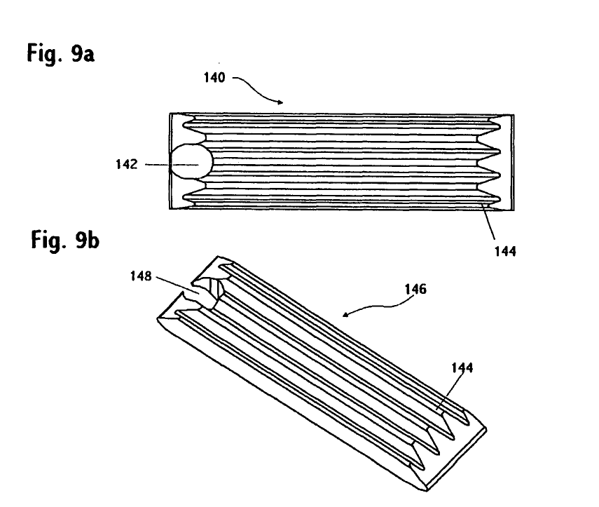

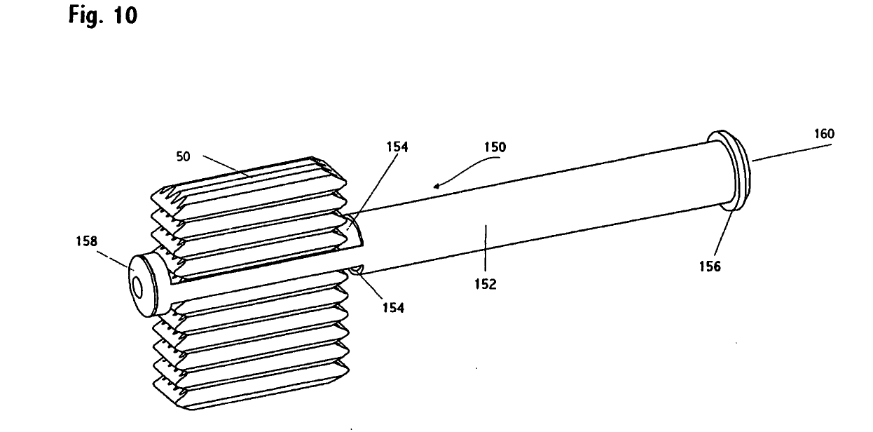

FIG. 9a illustrates another alternative configuration for a single plate with grooves and a closed bore at one end. Here, the plate 140 has several substantially parallel grooves 144 on its top and bottom surfaces, so as to facilitate convenient sliding of one plate on top (or beneath) another plate, keeping them aligned and preventing lateral relative motion. Optionally, at one of its tapered ends a bore is provided in order to facilitate hooking the plate to an introducing tool (not shown in the drawing) with matching hook, so that the plate is hooked onto the introducing tool while being delivered to its target position, and released when in position, allowing the introducing tool to be retracted. Further, the bore could be later re-hooked to retrieve plates from the plate assembly.

FIG. 9b illustrates another alternative configuration for a single plate 146, with grooves 144 and an open bore 148 at one end. The bore is opened at its side at the rim, so that the plate may be hooked onto a wire or a bar whose diameter tapers, the wider portion of the wire occupying substantial portion of the bore and prevented from slipping through the opening, whereas the narrow portion of the wire can slide out through the opening, for hooking onto or releasing the plate. Hooking the plate at the end of its longitude travel will add to the plate assembly stability.

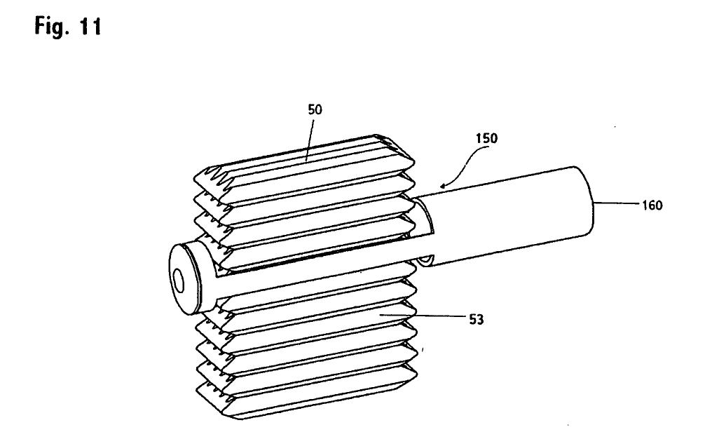

FIG. 10 illustrates a lead 150 for deploying a plate assembly in accordance with the present invention, with a flange 156. The lead serves to provide better control of the buildup of the plate assembly. The Lead 150 is basically a conduit 152 with an inlet at one end and two substantially opposite slits 154 at its other end, large enough to let a plate pass through it. Each plate 148 is introduced through the lead 150 from its rear end (with optional flange 156) and when it reaches the distal end 158, where the slots are located, it either drops down through the bottom slit (for example, in the case of the first plate introduced) or pushed up through the top slit, as more and more plates are piled up. Some plates will be pushed in between to previously adjacent plates pushing these plates away and squeezing in. Optionally, for the purpose of erecting a plate assembly within a vertebra, and introducing the lead through the pedicle, it is suggested that the length of the lead is calculated so that the inlet be left outside the vertebra. But this is not a requirement (see for example the shortened version of FIG. 11). The lead acts as a plate diverter so that when plates are inserted through the inlet, and advanced forward towards the distal end, their movement is perpendicularly diverted to protrude out of the slits and form the plate assembly.

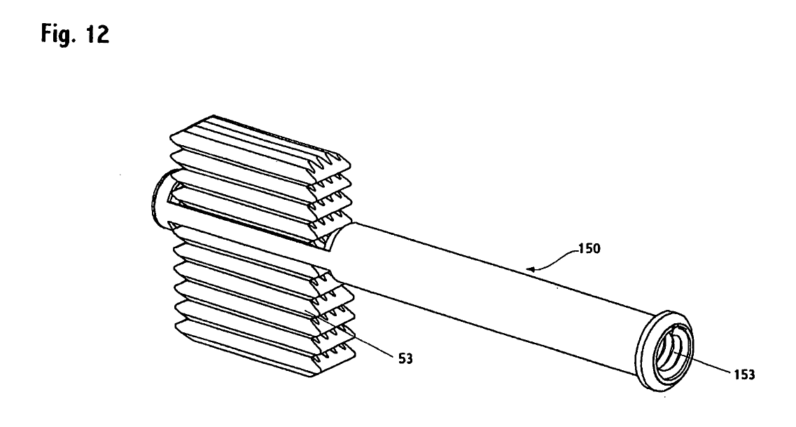

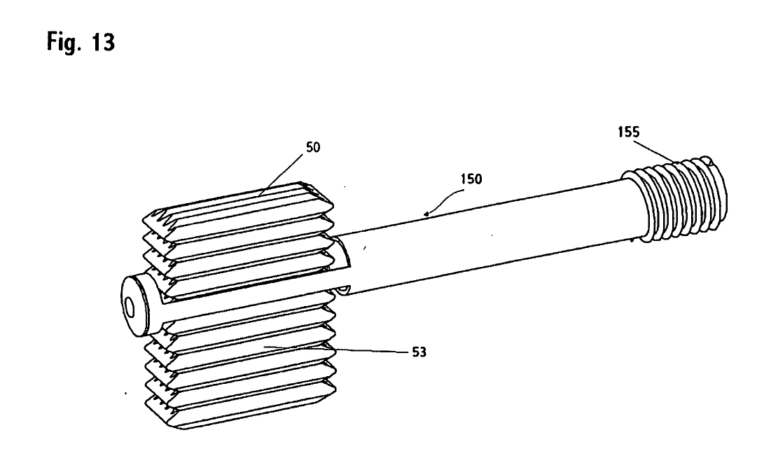

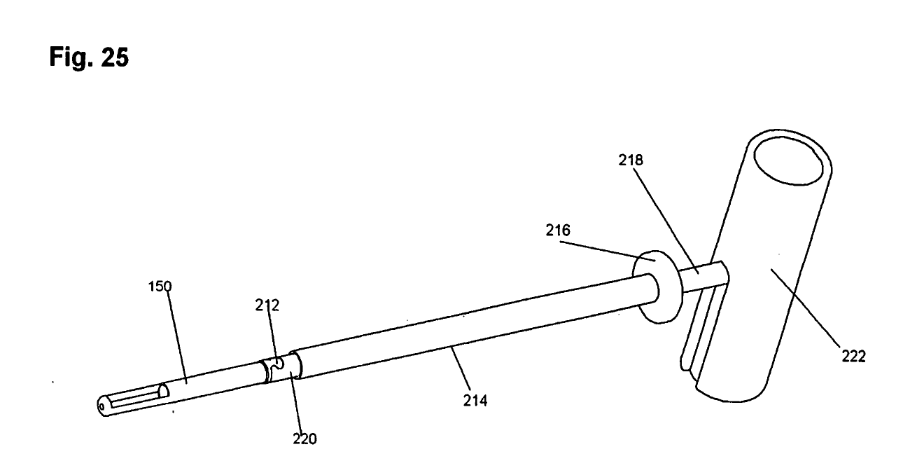

The flange 156 may serve to allow an introducing tool (such as the one shown in FIG. 25) to clasp it, advancing it while holding it firmly. The lead may also be attached to the introducing tool by way of screwing it into or onto the introducing tool (see FIGS. 12 and 13), or by employing any other method of attachment (see for example FIG. 25).

The introducing tool may introduce the plates through the lead, preferably one at a time.

The lead may include internal track on which the plate travels through, in order to maintain the desired orientation of the plate. Alternatively, the plate may be held in the right orientation by the introducing tool.

FIG. 11 illustrates a shortened lead for deploying a plate assembly in accordance with the present invention. Here the lead 150 is shorter than the one shown in FIG. 10, and therefore is fully inserted in the vertebra, or the treated bone.

The size of the lead may be provided in different sizes, according to its anticipated task and the size of the treated bone.

FIG. 12 illustrates a lead for deploying a plate assembly in accordance with the present invention, with internal thread. For introduction purposes it may be desired to use an introducing tool that can be temporarily attached to the lead, and released after the plate assembly 53 has been erected (or at any other desired time). For that end the lead of FIG. 12 has internal thread 153 at its inlet designed for matching external thread of the introducing tool. Similarly, FIG. 13 illustrates a lead for deploying a plate assembly in accordance with the present invention, with external thread 155. This external thread could be used to better secure the lead into the pedicle, fixating the posterior part of the vertebra to the vertebra body. The thread—internal or external—could later be used for attaching some other fixation device to the lead.

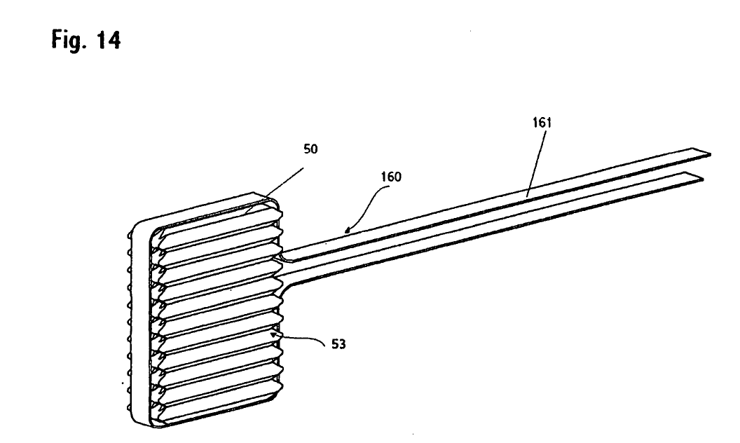

FIG. 14 illustrates a plate assembly with a packing strip 160 for packing the plate assembly in accordance with the present invention. The packing strip may be metallic or made from other strong and durable material yet flexible enough to allow reshaping as the plate assembly grows larger within. The packing strip holds the plate assembly together. Initially, the packing strip is introduced in a flat configuration (portion 161) and as plates are pushed through it bulges to allow the plate assembly to be built up. At the end of the introducing procedure, the strip may be cut somewhere along the residual area (161).

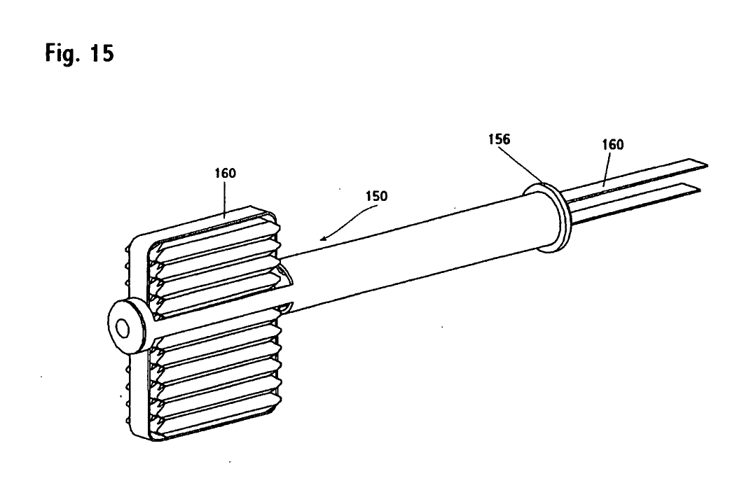

FIG. 15 illustrates a lead for deploying a plate assembly in accordance with the present invention, with a packing strip. Here the packing strip 160 is combined with the lead 150, passing through it. Initially, the packing strip is introduced in a flat configuration (see portion 161 at FIG. 14) and as the plates pile up the strip bulges out of the slits of the lead.

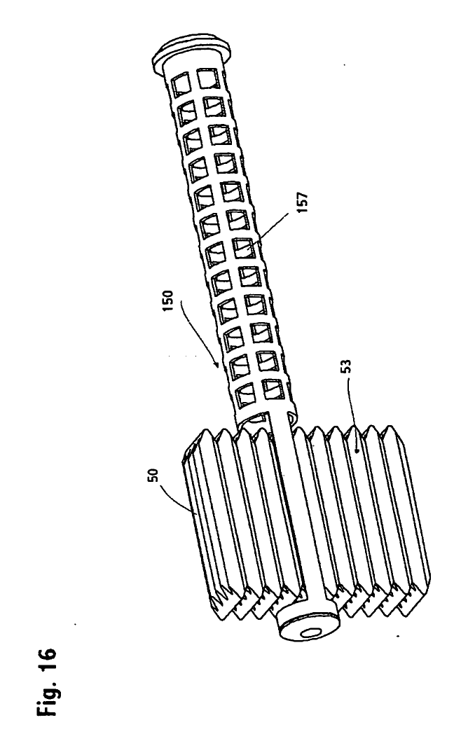

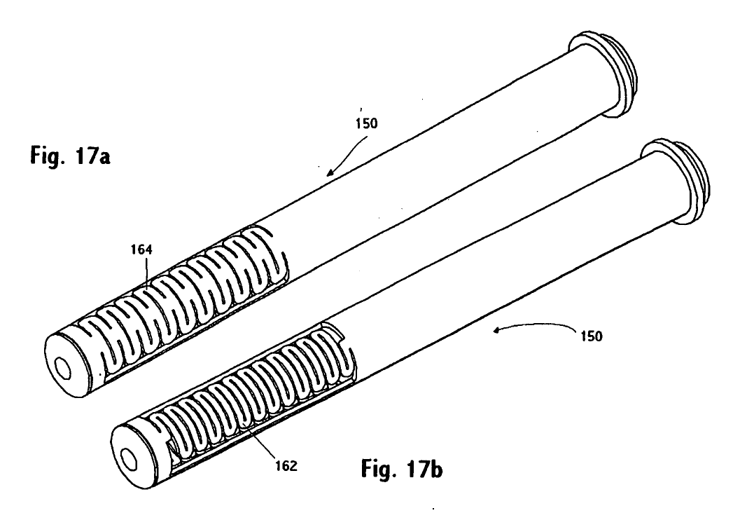

FIG. 16 illustrates a lead for deploying a plate assembly in accordance with the present invention, with openings 157 provided on the body of the lead for enhanced bone growth through/into the lead. The shapes and sizes of the spaces as well as their distribution along the lead may vary.

FIG. 17a illustrates a lead for deploying a plate assembly in accordance with the present invention, with integral deployable packing strips. Two opposite portions 164 at the distal end of the lead are carved, the shape of the carved portion preferably being entwined, to create a cage. When the plates are introduced through the lead into the cage, the internal force exerted on either carved portions causes the entwined carved portions to bulge out, serving as packing straps to the plate assembly formed within.

FIG. 17b illustrates another lead for deploying a plate assembly in accordance with the present invention, with integral deployable packing strip. The shape of the entwined carved portion here 162 is in the form of a curved strip.

The shapes of these deployable packing strips may vary, as long as they allow bulging of the plate assembly while effectively wrapping it.

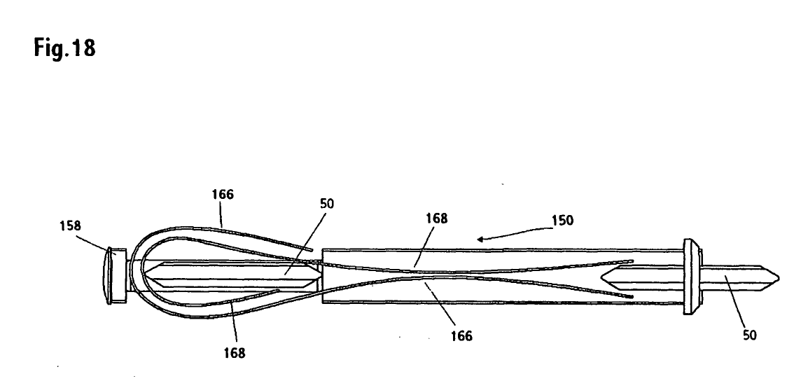

FIG. 18 illustrates a sectioned view of a lead for deploying a plate assembly in accordance with the present invention, with deployable packing strips. The lead 150 is provided with two internal straps 166, 168, which are long enough so that when the plate assembly is erected, one strap covers the plate assembly from its top whereas the other strap closes on the plate assembly from the bottom, the ends of the straps overlapping at the distal end 158 of the lead.

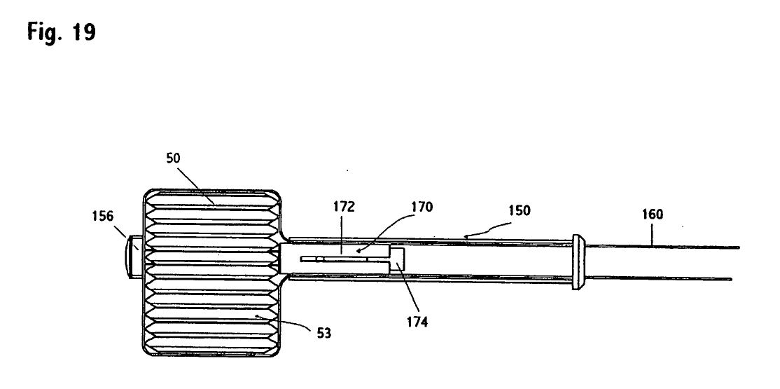

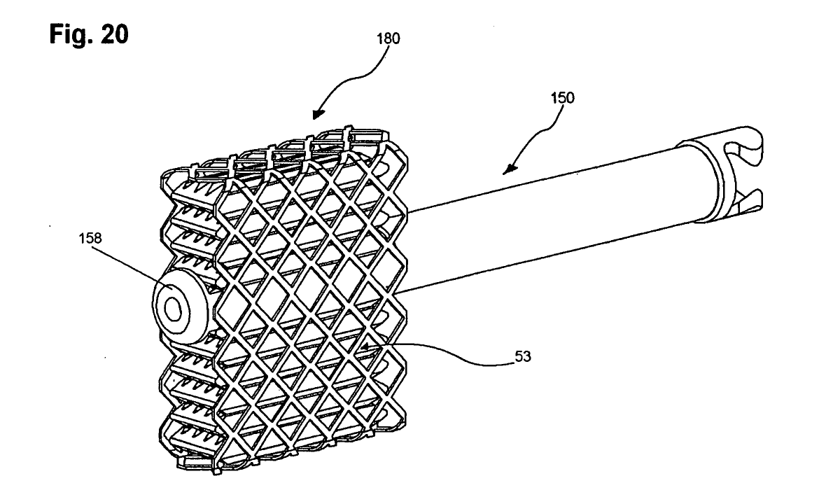

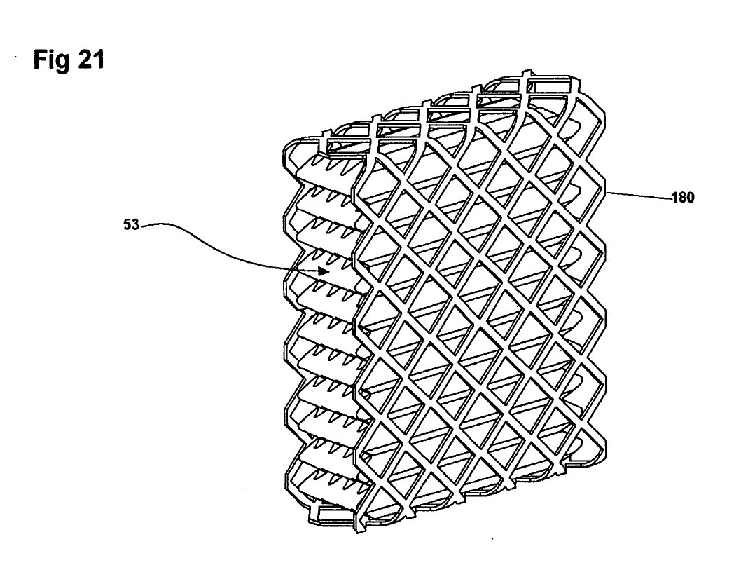

FIG. 19 illustrates a sectioned view of a lead for deploying a plate assembly in accordance with the present invention, with a packaging strip and a stopper. This is a modified version of the embodiment shown in FIG. 15. A stopper 170 is provided in the form of a plug device, here made up of two parts—a socket 172 and a plug 174, which is tapered. As the plug 174 is plugged into the socket 172 it exerts force pressing the packing strap onto the internal wall of the lead, effectively locking it in position.

FIG. 20 illustrates a lead 150 for deploying a plate assembly in accordance with another preferred embodiment of the present invention, with a deployable cage 180. The cage is initially cramped over the portion of the lead where the slits are and as the plates start to build-up and protrude from the lead the cage extends, enveloping the plate assembly 53. The cage may be manufactured from durable strong materials in a construction that is capable of expanding, such as shape memory alloys (like NiTi), steel or other materials. The deployable structure may in fact be a stent.

FIG. 21 illustrates a plate assembly with a deployable cage in the deployed state. Here the cage 180 is used independently of a lead, and is introduced into the treated bone in a cramped position. As the plates are introduced into it and a plate assembly rises, the cage expands to hold the erected plate assembly.

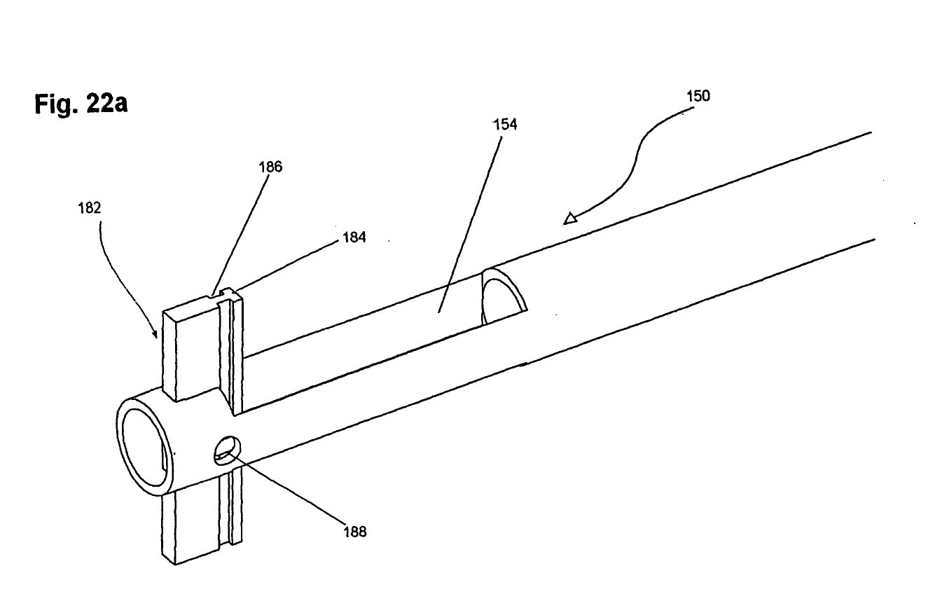

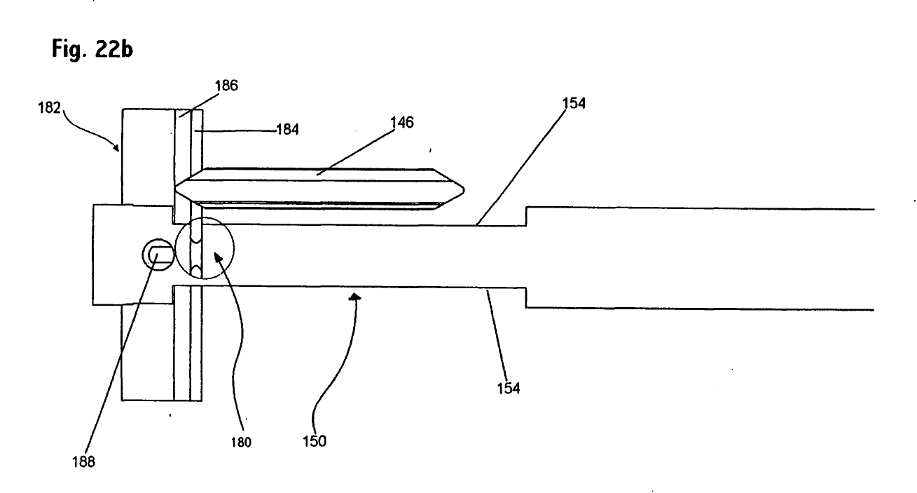

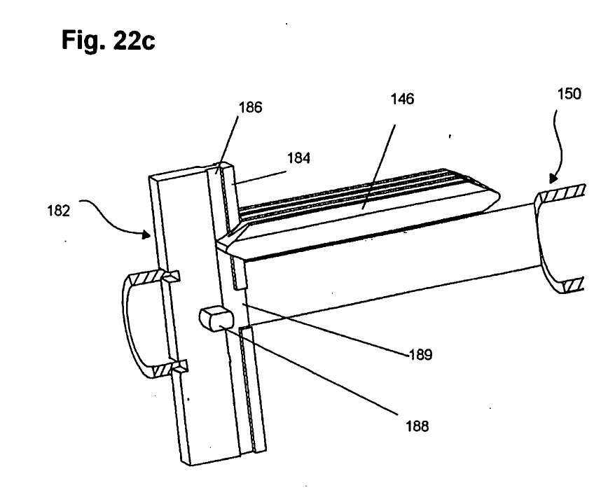

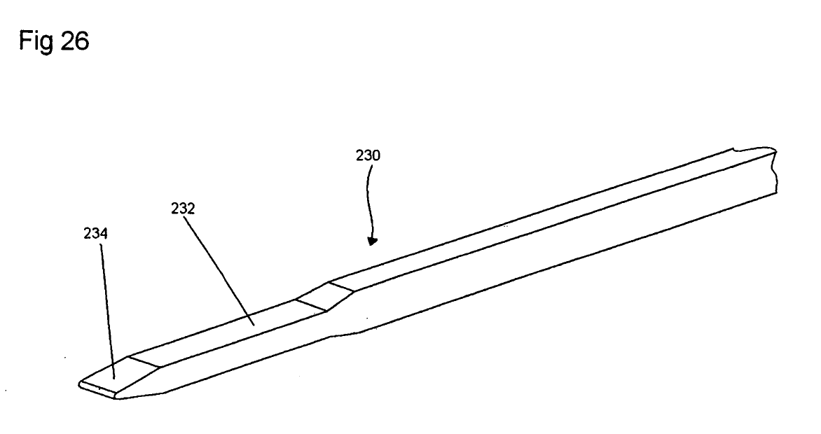

FIG. 22a illustrates a lead 150 for deploying a plate assembly in accordance with another preferred embodiment of the present invention, with a tiltable plate anchorage 182. The titlable plate anchorage in FIG. 22a is in the form of a blade having an elongated end presenting a T-shaped cross-section, with a narrow portion 186 and a wider portion 184. The blade is initially advanced through the lead in a horizontal position (the T-shaped end either facing downward or upward), and as it reaches the distal portion (where slits 154 are) it is flipped, using a tool (see for example the splitting tool shown in FIG. 26) or by a resilient mechanism incorporated in the lead or the anchorage blade (such as a spring) to an upright vertical position (as shown in the Figure). A central portion 189 of the T-shaped end is tapered (see FIGS. 22b & 22c) so as to allow plates having an open bore at their end (see FIG. 9b) to hook onto the blade's end. As they shift upwards or downwards along the anchorage blade, the wider portion 184 substantially occupies the bore, so that the plate cannot be released from the anchorage blade's end, thus providing additional stability to the plate assembly.

FIG. 22b illustrates a side view of the lead of FIG. 22a with a plate 146 anchored to the tiltable plate anchorage 182. The area 180 shows a transparent circle in the lead and reveals the tapered opening 189 in the central portion of the T-shaped end, allowing plates having an open bore at their end (see FIG. 9b) to hook onto the blade's end. The tiltable plate anchorage 182 may be pivotally attached to the lead at a pivot 188, preferably in the form of a projection snapped into its place inside a matching bore in the lead.

FIG. 22c illustrates a side view of a portion of the lead of FIG. 22a with the front side of the lead missing to allow understanding of how the plates anchor to the tiltable plate anchorage.

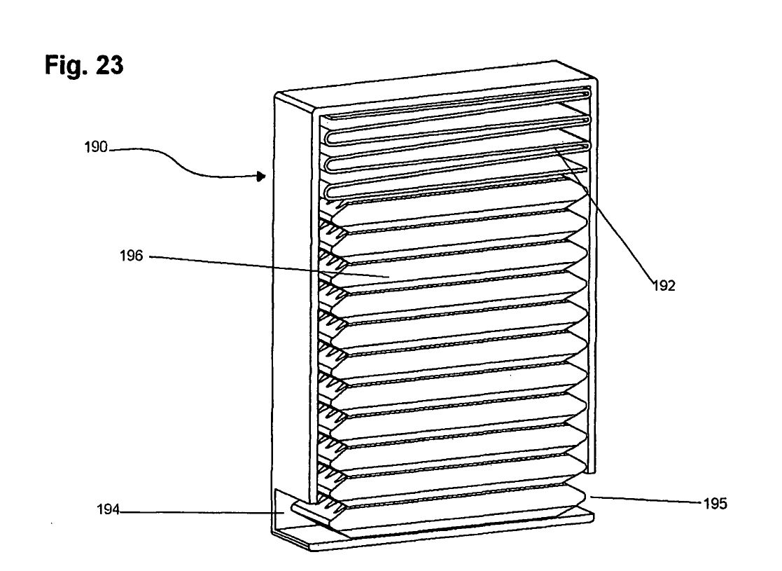

FIG. 23 illustrates a plate cartridge with vertically mounted plates in accordance with a preferred embodiment of the present invention. The cartridge 190 comprises a housing (the front wall of the housing is not shown in order to allow a view of the cartridge's contents) with an inlet 194 and outlet 195, capable of holding a predetermined number of plates 196—here stacked one on top of the other and pressed against a spring 192, which is aimed at pressing the plates towards the inlet/outlet openings. The inlet and outlet openings are substantially opposite each other so that a delivery tool may be inserted through the inlet and push a plate out through the outlet and towards the target position of the plate. The cartridge greatly simplifies the positioning procedure of the plate assembly, for it relieves the doctor or the technician from the need to check the orientation of each plate before insertion. The cartridge may also be used in corporation with an automated or semi-automated delivery device for delivering the plates to their target position within the treated bone.

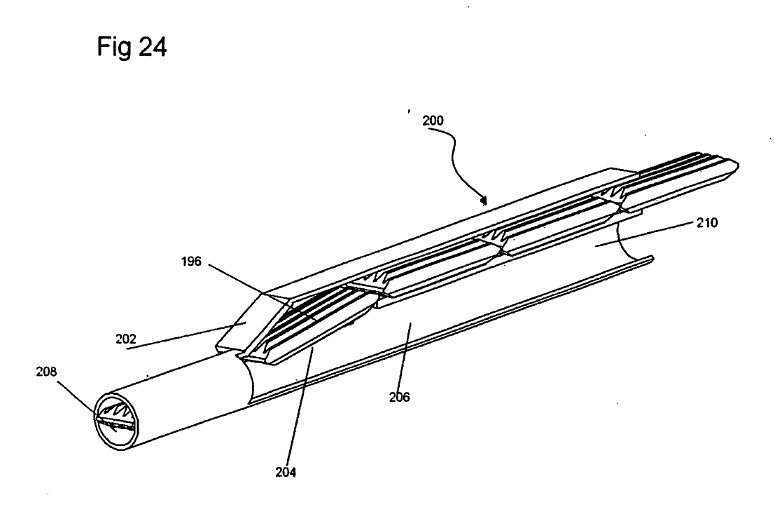

FIG. 24 illustrates another plate cartridge 200 with plates 196 arranged in a line, and provided with an introducing duct 206. Here the plates are arranged in a line and their housing 202,is adjacent an introducing duct 206, with an opening 204 to allow the plates, one at a time, to enter the introducing duct 206. An introducing tool (such as the delivery tool 46 of FIGS. 3 & 4, or the splitter 230 of FIG. 26, or a similar device) is inserted through the introducing duct inlet 210 and pushes the plate out through outlet 208. The introducing duct is preferably connected to a lead (such as those shown in the Figures) or is used independently for delivering plates to the treated bone.

FIG. 25 illustrates a delivery tool in accordance with a preferred embodiment of the present invention, with yet another preferred embodiment of a lead for deploying a plate assembly mounted on its tip. The delivery tool is an elongated tool used to hold the lead 150 and advance it towards its target destination within the treated bone. Here the tool comprises two coaxial pipes 214 (external pipe) and 218 (internal pipe). The internal pipe is provided at its distal tip 220 with a recess of a predetermined shape and the lead 150 is provided at its proximal end with a protrusion having a shape matching that of the recess so that the protrusion may rest within the recess thus coupling the lead to the introducing tool. In order to disengage the lead from the introducing tool a lateral relative movement between the tool and the lead is required. The external pipe 214 is used to prevent inadvertent disengagement by covering the distal end of the internal pipe when the lead is not yet at its final position, and the introducing tool is advancing it towards its destination. Once in position, the lead is released by pulling the external pipe 214 (by retracting ring 216 that is coupled to the external pipe for the sake of convenient gripping) over the internal pipe 218, so as to uncover the distal tip of the internal pipe and allow the disengagement of the lead. Knob 222 is provided at the proximal end of the internal pipe for a convenient grip of the tool. The length of the tool is predetermined to allow convenient use and handling of the proximal ends of the internal and external pipes outside the patient's body, while the distal ends are near or at the target location.

FIG. 26 illustrates a spacing tool for providing room and controlling the alignment of the plates of a plate assembly in accordance with a preferred embodiment of the present invention. The spacing tool 230 may be used for pushing the plates towards their target location through a lead or through an introducing duct. In a preferred embodiment of the spacing tool it is provided with a tapered end 232, preferably in the shape of a plate (with a wedge 234), so that it may be used to detect the need for insertion of more plates by estimating the space left for an additional plate. It is inserted after one or more plates were introduced into their position, and is used to probe the room left. If it may be pushed in easily this may indicate that there is still room for at least one more plate. Furthermore the spacing tool may be used to align the plates in position (if they are somewhat disorganized) by providing a splitting force that presses some plates upwards and some downwards. In a preferred embodiment of the spacing tool, it may be provided with a pressure sensor to sense and indicate the pressure on the plate assembly, thus indicating whether the plate assembly still requires additional plates.

The plates may be also arranged side by side (with the aspects previously referred to as “top” or “bottom” in the explanation hereinabove lying side by side laterally), to provide a lateral supporting construction.

By inserting a plurality of plates into the desired position within the bone or space previously occupied by intervertebral disc, it is possible to fill the space substantially with the plates for enhanced fixation.

Again, it is emphasized that these are merely several alternatives suggested. The features of the plates, and in particular the guiding features, may be designed in various ways, and a person skilled in the art could easily design other such guiding features that are different from the features described herein. However the scope of the present invention is not limited to the guiding features described herein in the specification and accompanying Figures, but rather defined by the appended Claims and their equivalents. It is also noted that it may be desired to mount plates of various types, sizes, or shapes on top of each other (for example using several plates shown in FIG. 6a in conjunction with one or several plates shown in FIG. 6c, etc.). Thus this invention further contemplates the creation of plates of various shapes and sizes having compatible locking mechanisms.

The top and bottom aspects may be designed in various shapes and textures (some of which are shown in the drawings.), and it is recommended to provide rough surfaces in order to enhance the friction between the plates and reduce their tendency to slide off each other.

In a preferred embodiment of the plate it is recommended to indicate the correct orientation on the plate, such as color coding (for example, assigning red to the upper surface and blue to the lower surface etc.), so that it is simple to use and does not require awkward scrutiny before use.

Optionally the plates may be provided in a cartridge, arranged in the correct orientation and ready for deployment by an automated or semi automated device.

The plates may be provided in various designs, such as straight, laterally curved, different elevations etc., according to the physical features sought. In a preferred embodiment of the present invention it is suggested to build two such vertebral modular implant support assemblies that form two walls with an angle between them, determined by the different pedicular entry angles (see FIG. 1). In another preferred embodiment it is suggested to couple two vertebral modular implant support assemblies at their adjacent ends.

The plates may be made from a rigid biocompatible material, for example metals such as titanium and it's alloys, stainless steel alloys e.g., steel 316, processed foil, hydroxyapatite, or material coated with hydroxyapetite, plastics (polimeric materials), silicon, composite materials (such as carbon-fiber), hardened polymeric materials e.g., polymethylmetacrylate (PMMA), ceramic materials, coral material. The plate may be covered with other substance encouraging bone growth on the implant (such as bone morphogenic protein). In yet another preferred embodiment the plates may be covered with medication substances, such as antibiotics, or slow releasing medication, such as chemotherapy substances, for long-term therapy. If it is desired to implant the vertebral modular implant support assembly in a magnetic resonance imaging (MRI) procedure the plates should be made from non-ferrous materials.

Other coating, such as lubricants for improved sliding of the plates into their target position, or coating materials that sublime or react to form a solid conglomerate, may be added too. Different coatings may be combined if compatible and beneficial.

It is noted that in particular cases it may be enough to implant only one plate without adding additional plates on top or next to that plate.

Present research contemplates development of materials that will be implantable within a bone and during the course of time give way (dissolve/degrade—biodegradable material) to bone material. The present invention may be implemented with such materials as well.

The method described herein is minimally invasive and as such has special appeal, for it substantially minimizes surgery-related infection risks, reduces the surgical procedure steps (and thus the costs involved), and shortens healing and recovery times for the patient.

It should be clear that the description of the embodiments and attached Figures set forth in this specification serves only for a better understanding of the invention, without limiting its scope.

It should also be clear that a person skilled in the art, after reading the present specification could make adjustments or amendments to the attached Figures and above described embodiments that would still be covered by the following Claims and their equivalents.

1. A modular reconstructing and supporting assembly for reconstructing and supporting a diseased or fractured bone or within a space previously occupied by a diseased intervertebral disc, the assembly comprising: a plurality of plates adapted to be cooperatingly inserted into the bone, at least one of said plates arranged adjacently to another plate within said bone or space, to construct scaffolding for forming a supporting prosthesis.

2. The assembly of claim 1, wherein at least one of said plates having at least two substantially opposite aspects with interlocking features designed to facilitate interlocking of adjacent plates so as to prevent or restrain relative movement therebetween.

3. The assembly of claim 2, wherein the opposite aspects of the plate are inclined with respect to each other.

4. The assembly of claim 2, wherein one of said aspects is provided with at least one longitudinal protrusion and the opposite aspect is provided with at least one corresponding longitudinal recess designed to receive a longitudinal protrusion of an adjacent plate.

5. The assembly of claim 2, wherein one aspect is provided with at least one lateral protrusion and the opposite aspect is provided with at least one corresponding lateral recess designed to accommodate a lateral protrusion of an adjacent plate.

6. The assembly of claim 2, wherein one aspect is provided with at least one longitudinal protrusion and at least one lateral protrusion and the opposite aspect is provided with at least one corresponding longitudinal recess designed to accommodate a longitudinal protrusion of an adjacent plate, and with at least one corresponding lateral recess designed to accommodate a lateral protrusion of an adjacent plate.

7. The assembly of claim 2, wherein the interlocking features include at least one recess on one aspect and at least one corresponding projection on the other aspect, so that the projection of one plate is accommodatable in the recess of an adjacent plate.

8. The assembly of claim 7, wherein the recess further comprises a rim adapted for retaining the projection of an adjacent plate, for preventing or restraining relative displacement therebetween.

9. The assembly of claim 8, wherein the rim extends along a portion of the circumference of the recess, allowing leveled sliding in of the projection of the adjacent plate.

10. The assembly of claim 1, wherein at least one of said plurality of plates is curved.

11. The assembly of claim 1, wherein the plate is provided with at least one tapered end, for facilitating plate guidance and positioning between two adjacent plates.

12. The assembly of claim 11, wherein the tapered end is in the form of a wedge.

13. The assembly of claim 1, wherein the plate is made from or coated with biocompatible material.

14. The assembly of claim 1, wherein the plate is made from material selected from a group consisting of metal, titanium, titanium alloy, stainless steel alloys, steel 316, processed foil, hydroxyapatite, material coated with hydroxyapetite, plastics, silicon, composite materials, carbon-fiber, hardened polymeric materials, polymethylmetacrylate (PMMA), ceramic materials, coral material, or a combination thereof.

15. The assembly of claim 1 wherein at least one of said plates is coated with hydroxyapetite

16. The assembly of claim 1, wherein the plate is covered with a bone growth encouraging substance.

17. The assembly of claim 1 wherein said plate being is coated with bone morphogenic protein.

18. The assembly of claim 1, wherein the plate is coated with medication.

19. The assembly of claim 1, wherein said plate is coated with a substance selected from the group consisting of antibiotics, slow releasing medication, chemotherapy substances, or a combination thereof.

20. The assembly of claim 1, wherein the plate comprises non-ferrous material.

21. The assembly of claim 1, wherein the plate is coated with lubricating material to facilitate sliding the plates into a desired position.

22. The assembly of claim 1, wherein the plate is coated with coating materials that sublime or react to form a solid conglomerate.

23. The assembly of claim 1, wherein the plate is substantially disc-shaped.

24. The assembly of claim 23, wherein the plate is provided with a groove adapted to be engaged by a holding tool.

25. The assembly of claim 23, further comprising a pin protruding from at least one of said plates, to facilitate placement of said plate.

26. The assembly of claim 1, wherein at least one of said plates having a rough external surface.

27. The assembly of claim 1, wherein the plate is provided with a plurality of substantially parallel grooves, so as to facilitate sliding of one plate adjacent another such plate.

28. The assembly of claim 1, wherein a bore is provided on the plate to facilitate hooking of the plate onto an introducing tool and releasing it when it is positioned at a desired location.

29. The assembly of claim 1, wherein the plate is provided with a bore with open rim.

30. The assembly of claim 1, further comprising a lead in the form of a conduit with a proximal end and a distal end, the conduit having an inlet at the proximal end and two substantially opposite slits about the distal end, so that when plates are inserted through the inlet and advanced towards the distal end, some plates protrude out of the slits to form the plate assembly.

31. The assembly of claim 30, wherein the lead is provided with thread at its proximal end.

32. The assembly of claim 31, wherein the thread is internal.

33. The assembly of claim 31, wherein the thread is external.

34. The assembly of claim 30, wherein a packing strip is provided in the lead to hold the plate assembly together.

35. The assembly of claim 34, further provided with a stopper in the form of a plug that plugs into the lead holding sides of the packing strap against the lead so as to lock the strap in position.

36. The assembly of claim 30, wherein the lead is provided with spaces designed to encourage bone growth into it.

37. The assembly of claim 30, wherein the slits are carved into the lead in an entwining form so as to produce portions that may bulge outwardly, for holding the plate assembly when erected.

38. The assembly of claim 37, wherein the entwined form consists of a curved strip.

39. The assembly of claim 30, wherein two straps are further provided within the lead, long enough so that when the plate assembly is erected, one strap covers the plate assembly from one side whereas the other strap closes on the plate assembly from another opposite side, portions of the straps overlapping at the distal end.

40. The assembly of claim 1, further provided with a crampable deployable cage for hosting the plate assembly when erected.

41. The assembly of claim 40, wherein the cage is a stent.

42. The assembly of claim 1, provided in a cartridge.

43. The assembly of claim 42, wherein the cartridge comprises a housing for hosting a plurality of plates stacked one on top of each other, with an inlet and outlet, the inlet and outlet substantially opposing each other, and a resilient member for pressing plates against the outlet so as to allow convenient drawing of a plate from the cartridge.

44. The assembly of claim 42, wherein the cartridge comprises an elongated housing for hosting a plurality of plates arranged in a line, with an adjacent introducing duct, the cartridge provided with an opening into the introducing duct so that one plate at a time may be inserted into the introducing duct and advanced through the duct to a target location using an introducing tool.

45. A lead device for introducing and supporting a plate assembly made of stacked plates, the lead comprising a conduit with a proximal end and a distal end, the conduit having an inlet at the proximal end and two substantially opposite slits about the distal end, so that when plates are inserted through the inlet and advanced towards the distal end, some plates protrude out of the slits to form the plate assembly.

46. The device of claim 45, further comprising thread at its proximal end.

47. The device of claim 46, wherein the thread is internal.

48. The device of claim 46, wherein the thread is external.

49. The device of claim 45, wherein a packing strip is provided in the lead to hold the plate assembly together.

50. The device of claim 49, further provided with a stopper in the form of a plug that plugs into the lead holding sides of the packing strap against the lead so as to lock the strap in position.

51. The device of claim 45, wherein the lead is provided with spaces designed to encourage bone growth into it.

52. The device of claim 45, wherein it is further provided with a crampable deployable cage for hosting the plate assembly when erected.

53. The device of claim 52, wherein the cage is a stent.

54. The device of claim 45, wherein the slits are carved into the lead in an entwining form so as to produce portions that may bulge outwardly, for holding the plate assembly when erected.

55. The device of claim 54, wherein the entwined form consists of a curved strip.

56. The device of claim 45, wherein two straps are further provided within the lead, long enough so that when the plate assembly is erected, one strap covers the plate assembly from one side whereas the other strap closes on the plate assembly from another opposite side, portions of the straps overlapping at the distal end.

57. The device of claim 45, further provided with a tiltable plate anchorage for anchoring plates to it for improved stability of the plate assembly.

58. The device of claim 57, wherein the tiltable plate anchorage is in the form of a blade having an elongated end presenting a T-shaped cross-section, with a narrow portion and a wider portion, the blade capable of being initially advanced through the lead in a horizontal position, and as it reaches the distal portion it is capable of flipping to an upright vertical position.

59. The device of claim 58, wherein a central portion of the elongated end presenting a T-shaped cross-section is tapered so as to allow plates having an open bore at their end to be hooked onto the end, and when the plates shift upwards or downwards along the anchorage blade, the wider portion substantially occupies the bore, so that the plate cannot be released from the anchorage blade, thus providing additional stability to the plate assembly.

60. A delivery tool for delivering a device as claimed in claim 45 into a diseased or fractured bone or within a space previously occupied by a diseased intervertebral disc, the delivery tool comprising two coaxial pipes, one internal pipe and one external pipe, the external pipe adapted to be shifted over the internal pipe so as to cover the latter or expose it, so that an engagement means located at a distal tip of the internal pipe is engaged when the external pipe covers the distal end of the internal pipe and disengaged when the distal end of the internal pipe is exposed.

61. The tool of claim 60 wherein the internal pipe is provided at the distal end with a recess of a predetermined shape so as to accommodate a matching protrusion of the device thus coupling the device to the delivery tool.

62. A spacing tool for spacing and evaluating the spacing between adjacent plates of the assembly claimed in claim 1, the spacing tool comprising a rod with a tapered end.

63. The spacing tool of claim 62, wherein the tapered end is provided with a wedge.

64. The assembly of claim 1, wherein a packing strap is provided to hold the plate assembly together when erected.

65. A plate for use in conjunction with at least another one of a plurality of other plates in a modular reconstructing and supporting assembly for reconstructing and supporting a diseased or fractured bone or within a space previously occupied by a diseased intervertebral disc of a patient, the plate sized small enough to be suitable for separate insertion into the bone or the space and arrangement with the other plates adjacently to construct scaffolding, so as to provide a supporting prosthesis.

66. A method for reconstructing and supporting within a diseased or fractured bone or within a space previously occupied by a diseased intervertebral disc the method comprising:
inserting a plurality of plates into the bone
arranging said plates adjacent one another, within the bone or space, to construct a support scaffolding.

67. The method of claim 66 further comprising the steps of delivering each plate separately into the bone using low profile delivery means, through a small incision in the skin of the patient, and arranging adjacent plates on top of each other.

68. The method of claim 67, wherein the delivery means comprises a canula and a rod with which the plates are each advanced through the canula.

69. The method of claim 68, wherein the rod is provided with holding means to hold the plates.

70. The method of claim 66, wherein the bone is a vertebra and the plates are inserted through a bore drilled into the body of the vertebra through a pedicle of the vertebra.

71. The method of claim 70, wherein the diameter of the bore is in a range between 4 to 8 mm.

72. The method of claim 66, wherein at least one of said plates has at least two substantially opposite aspects with interlocking features designed to facilitate interlocking of adjacent plates, for preventing or restraining relative displacement therebetween.

73. The method of claim 72, wherein one aspect is provided with at least one longitudinal protrusion and the opposite aspect is provided with at least one corresponding longitudinal recess designed to accommodate the longitudinal protrusion of an adjacent plate.

74. The method of claim 72, wherein one aspect is provided with at least one lateral protrusion and the opposite aspect is provided with at least one corresponding lateral recess designed to accommodate the lateral protrusion of an adjacent plate.

75. The method of claim 72, wherein one aspect is provided with at least one longitudinal protrusion and at least one lateral protrusion and the opposite aspect is provided with at least one corresponding longitudinal recess designed to accommodate the longitudinal protrusion of an adjacent plate, and with at least one corresponding lateral recess designed to accommodate the lateral protrusion of an adjacent plate.

76. The method of claim 72, wherein the interlocking features include at least one recess on one aspect and at least one corresponding projection on the other aspect, so that the projection of one plate is accommodated in the recess of an adjacent plate.

77. The method of claim 66, wherein at least one of said plurality of plates is provided with at least one tapered end, to facilitate positioning the plate between two adjacent plates.

78. The method of claim 66, wherein at least one of said plurality of plates is substantially disc-shaped.

79. The method of claim 66, wherein at least one of said plurality of plates is further provided with a protruding pin, adapted to facilitate holding the plate by a delivering tool.

80. The method of claim 66, wherein the plates are inserted bilaterally constructing at least two scaffolding structures within a vertebral body.

81. The method of claim 66, wherein the plates are positioned one on top of the other.

82. A method for reconstructing and supporting within a diseased or fractured bone or within a space previously occupied by a diseased intervertebral disc the method comprising:
providing a plurality of plates adapted to be separately inserted into the bone and arranged adjacently within the bone or space to construct scaffolding for providing support;
providing delivery means having low profile for delivering each plate through a small incision in the skin of the patient and into the bone or disc;
delivering each plate separately into the bone;
arranging the plates one adjacent the other.
